(19) United States
(12) Patent Application Publication
(10) Pub. No.: US 2011/0217369 A1
(43) Pub. Date: Sep. 8, 2011

(54) FENOFIBRATE COMPOSITIONS

(75) Inventors: Amit Gupta, Bijnor (IN); Nidhi Singh, Gurgaon (IN); Romi Barat Singh, Varanasi (IN)

(73) Assignee: RANBAXY LABORATORIES LIMITED, New Delhi (IN)

(21) Appl. No.: 12/875,973

(22) Filed: Sep. 3, 2010

(30) Foreign Application Priority Data
Sep. 3, 2009 (IN) 1820/DEL/2009

(51) Int. Cl. A61K 9/48; A61K 31/235; A61K 9/00; A61K 9/20; A61P 3/06; A61P 9/00; B29C 43/02; B65B 1/04

(52) U.S. Cl. 424/452; 514/543; 424/400; 424/465; 264/122; 53/428

(57) ABSTRACT

The present invention relates to a fenofibrate composition comprising: fenofibrate, a surfactant, a hydrophilic polymer and one or more anti-foaming agents. The invention also relates to a novel process for preparing said composition that has enhanced dissolution and absorption characteristics.

### FIELD OF THE INVENTION

The present invention relates to a fenofibrate composition comprising fenofibrate, a surfactant, a hydrophilic polymer and one or more anti-foaming agents, and processes for preparing the same.

### BACKGROUND OF THE INVENTION

Fenofibrate belongs to the fibrate family, has been known for many years as a medicinal active principle because of its efficacy in lowering the blood triglyceride and cholesterol levels. Thus, fenofibrate is widely recommended in the treatment of hyperlipidemia and hypercholesterolemia.

Fenofibrate is a prodrug that is absorbed and then hydrolyzed by tissue and plasma esterases to fenofibric acid, its active metabolite or active species. Fenofibric acid, responsible for the pharmacological activity, has a plasma half-life of about 20 hours. Fenofibrate suffer from the disadvantage of being poorly soluble in an aqueous medium, thus having an insufficient dissolution profile and, consequently, poor bioavailability within the organism, following oral administration. The therapeutic dose required to be administered must thus be increased in order to obviate this disadvantage. This particularly applies to numerous hypolipemiant active ingredients, such as those belonging to the fibrate family. Indeed, due to its poor hydrosolubility, fenofibrate is poorly absorbed in the digestive tract and consequently its bioavailability is incomplete, irregular and often varies from one person to another.

To improve the dissolution profile of fenofibrate and its bioavailability, thereby reducing the dose requiring to be administered, it would be useful to increase its dissolution so that it could attain a level close to 100%.

Fenofibrate formulations have been prepared in several different ways in order to improve its bioavailability.

WO 82/01649 discloses a fenofibrate formulation having granules that are comprised of a neutral core that is a mixture of saccharose and starch. The neutral core is covered with a first layer of fenofibrate, admixed with an excipient and with a second microporous outer layer of an edible polymer.

European Patent No. EP 0 724 877 describes fenofibrate powder co-micronized with a wetting agent in association with a Vitamin E component (tocopherol and/or its organic acid ester) for treating or preventing disorders associated with lipoprotein oxidation.

U.S. Pat. No. 4,800,079 describes a medicinal composition in the form of granules with controlled release of fenofibrate. Each granule includes an inert core, a layer based on fenofibrate and a protective layer. Fenofibrate is present in the form of crystalline microparticles of dimensions not greater than 30 μm.

U.S. Pat. No. 4,961,890 describes a process for preparing a controlled release formulation containing fenofibrate in an intermediate layer in the form of crystalline microparticles (less than 30 μm in diameter) within a multilayer layer inert matrix.

European Patent No. EP 0 904 781 describes a process for making granules of a solid dispersion of a disintegrant in molten fenofibrate by blending a solid dispersing agent into molten fenofibrate, cooling and solidifying the bulk mixture in a tray, and then milling the solid through a screen to produce granules. Disintegrants include polymers such as starch, croscarmellose sodium, sodium starch glycolate, and crospovidone. Such disintegrants are slow to swell and dissolve in aqueous media. Furthermore, when crosslinked as in the case of crospovidone, a polymeric disintegrant will not be uniformly dissolved in molten drug but rather at best will form micro-domains in molten fenofibrate. In addition, polymeric materials can exhibit phase separation phenomena when distributed in a substance with which there is not complete compatibility.

European Patent No. EP 0 330 532 discloses a method for improving bioavailability of fenofibrate. This patent describes the effect of co-micronizing fenofibrate with a surfactant, for example sodium laurylsulfate in order to improve fenofibrate solubility and thereby increase its bioavailability. This patent teaches that co-micronizing fenofibrate with a solid surfactant improves fenofibrate bioavailability to a much greater extent than the improvement that would be obtained either by adding a surfactant, or through solely micronizing the fenofibrate, or, yet again, through intimately mixing the fenofibrate and surfactant, micronized separately.

The process of EP 0 033 532 leads to a new dosage form in which the active ingredient, co-micronized with a solid surfactant, has improved fenofibrate dissolution, and thus increased bioavailability, which makes it possible, for a given level of effectiveness, to decrease the daily dose of the medicament: respective 67 mg and 200 mg instead of 100 mg and 300 mg.

However, the preparation method in that patent is not completely satisfactory in as much as it does not lead to complete bioavailability of the active ingredient, and suffers from several disadvantages. The technique of co-micronizing fenofibrate with a solid surfactant improves dissolution of the active ingredient, but still it's incomplete.

Our co-pending Indian Patent Application No. 2752/DEL/2006 discloses a novel process to prepare fenofibrate composition. In the process, fenofibrate, surfactant and hydrophilic polymer mixture is dissolved in water-organic solvent mixture and sprayed over the inert carriers. The formulation is highly bioavailable since the fenofibrate is already in the solubilized form.

In the present invention, the process of preparing fenofibrate composition involves simple techniques and low cost production. Also, the composition of the present invention has an enhanced dissolution and absorption characteristics that provides complete and constant bioavailability to the patients.

### SUMMARY OF THE INVENTION

In one aspect, the present invention provides a fenofibrate composition comprising: fenofibrate, a surfactant, a hydrophilic polymer and one or more anti-foaming agents, wherein said fenofibrate is present in a concentration from 25 to 50% by weight of the composition.

In another general aspect, there is provided a fenofibrate composition in the form of capsules or tablets, comprising:
(i) fenofibrate, in a concentration from 25 to 50% by weight of the composition,
(ii) a surfactant, in a concentration from 0.05 to 5% by weight of the composition,
(iii) a hydrophilic polymer, in a concentration from 15 to 20% by weight of the composition, and
(iv) one or more anti-foaming agents, in a concentration from 0.05 to 5% by weight of the composition.

Embodiments of the composition may include one or more of the following features. For example, the fenofibrate used in the composition is in the micronized form and more particularly, micronized fenofibrate having d_{0.5} between 1 μm to 20 μm and d_{0.9} between 2 μm to 40 μm.

In another embodiment, the surfactant utilized is an anionic surfactant and more particularly, the surfactant is sodium lauryl sulfate.

In another aspect, the present invention provides a process for preparing a fenofibrate composition comprising the steps of:
(i) preparing a homogenized dispersion comprising fenofibrate, a surfactant, a hydrophilic polymer and one or more anti-foaming agents,
(ii) spraying the dispersion of step (i) over one or more inert carriers to form granules,
(iii) drying the granules of step (ii) and optionally, blending with one or more pharmaceutically acceptable excipients, and
(iv) filling the mixture of step (iii) into capsules or compressing into tablets.

In yet another embodiment, the inert carriers include one or more pharmaceutically acceptable materials of water-soluble, water-insoluble, and combinations thereof. Particularly the carrier used is pregelatinized starch.

The pharmaceutically acceptable excipients may be one or more of fillers, binders, disintegrants, lubricants, glidants, coloring agents and flavoring agents.

According to one of the embodiments, the fenofibrate composition prepared in the said process may be optionally further coated with one or more film forming agents.

It is yet another aspect to provide a method of treating hyperlipidaemic or dyslipidimic conditions by administering to a person in need thereof, a pharmaceutical composition of fenofibrate. The composition may further include one or more other antilipidaemic agents.

The details of one or more embodiments of the invention are set forth in the description below. Other features, objects, and advantages of the invention will be apparent from the description and claims.

### DETAILED DESCRIPTION OF THE INVENTION

The present invention relates to a fenofibrate composition in the form of capsules or tablets that has enhanced dissolution and absorption characteristics. The enhanced dissolution property is achieved primarily by the process that involves dispersing fenofibrate along with surfactant and hydrophilic polymer in a solution and spraying the dispersed fenofibrate over the inert carriers for preparing the granules. This technique advantageously has enhanced dissolution and absorption characteristics as it provides the active fenofibrate already in the dispersed form as soon as it enters the gastro-intestinal region.

In our co-pending Indian Patent No. 2752/DEL/2006 dated. Dec. 21, 2006 (this application is incorporated herein by reference in its entirety), fenofibrate composition is prepared by spraying a solution comprising fenofibrate, surfactant and hydrophilic polymer, over inert carrier materials. The solvent used to prepare the solution is water-organic solvent mixture. However, usage of organic solvent restricts the acceptability of this process due to environment concern.

On the contrary, the present invention does not involve usage of any organic solvents in the entire process. Formulating a highly bioavailable fenofibrate composition without using any organic solvent is complicated, due to process related difficulties. During the process when the mixture of fenofibrate, surfactant and hydrophilic polymer is homogenized in aqueous solvent, large amount of froth develops. This froth generation would lead to improper drug distribution throughout the drug solution mixture and poses difficulty in spraying. The present invention overcomes this trouble by incorporating correct proportion of anti-foaming agent, without hindering the bioavailability of fenofibrate.

The invention relates to a fenofibrate composition in the form of capsules or tablets comprising: fenofibrate, a surfactant, a hydrophilic polymer and one or more anti-foaming agents, wherein said composition is prepared by the process comprising the steps of:
(i) preparing a homogenized dispersion comprising fenofibrate, a surfactant, a hydrophilic polymer and one or more anti-foaming agents,
(ii) spraying the dispersion of step (i) over one or more inert carriers to form granules,
(iii) drying the granules of step (ii) and optionally, blending with one or more pharmaceutically acceptable excipients,
(iv) filling the mixture of step (iii) into capsules or compressing into tablets.

The fenofibrate used in the composition is in the micronized form, particularly the micronized fenofibrate having d_{0.5} between 1 μm to 20 μm and d_{0.9} between 2 μm to 40

The concentration of fenofibrate used in the composition typically ranges from 25 to 50% by weight of the composition.

The term “d_{0.5}”, as used herein, with reference to the size of micronized fenofibrate, indicates that about 50 volume % of particles measured have a size less than the defined d_{0.5} value and that about 50 volume % of particles measured have a size greater than the defined d_{0.5} value. The term “d_{0.9}”, as used herein, indicates that about 90 volume % of particles measured have a size less than the defined d_{0.9} value and that about 10 volume % of particles measured have a size greater than the defined d_{0.9} value.

The solvent used for preparing fenofibrate dispersion is water.

Surfactants help in increasing the solubility of fenofibrate and thereby increase the dissolution rate. The term “surfactant” is used in its conventional sense throughout this invention. Suitable surfactants can be anionic, cationic, zwitterionic and nonionic surfactants. Preferably, the compositions include at least one anionic surfactant. Suitable anionic surfactants include, but are not limited to, alkyl sulfonates, alkyl phosphates, alkyl phosphonates, potassium laurate, sodium lauryl sulfate or sodium dodecylsulfate, alkyl polyoxyethylene sulfates, docusate sodium, dioctyl sodium sulfosuccinate, phosphatidyl glycerol, phosphatidylinositol, diphosphatidylglycerol, phosphatidyl inosine, phosphatidylserine, phosphatidic acid and their salts, cholic acid and other bile acids (e.g., cholic acid, deoxycholic acid, glycocholic acid, taurocholic acid, glycodeoxycholic acid), and salts thereof (e.g., sodium deoxycholate, etc.).

The concentration of surfactant used typically ranges from 0.05% to 5% by weight of the composition.

Hydrophilic polymers present along with the fenofibrate dispersion improve solubility of fenofibrate. These include, but are not limited to, pharmaceutically acceptable materials like starch, gums, alginates, polysaccharides, polyvinylpyrrolidone, polyethylene glycol, acrylic acid derivatives, and cellulose derivatives like hydroxypropyl cellulose, hydroxypropyl methylcellulose, hydroxyethylcellulose, hydroxymethylcellulose, carboxymethylcellulose, methylcellulose, sodium carboxy methylcellulose, and mixtures thereof. Particularly, the hydrophilic polymer used is a cellulose derivative and more particularly it is hydroxypropyl methylcellulose.

The concentration of hydrophilic polymer used typically ranges from 15% to 20% by weight of the composition.

Anti-foaming agents used during the process prevent or reduce frothing or foaming during manufacture. These include, but are not limited to, simethicone, polyglycol, silicon oil, and mixtures thereof. Particularly the anti-foaming agent is simethicone.

The concentration of anti-foaming agent used typically ranges from 0.05% to 5% by weight of the composition.

The homogenized dispersion containing fenofibrate can be sprayed using coating equipment known in the pharmaceutical arts, such as fluidized bed coaters (Wurster coaters or top-sprayers), pan coaters and rotary granulators. The spray nozzle can be placed in the top, side walls or the bottom of the spraying chamber and the chamber can be provided with more than one nozzle.

The inert carriers may be fluidized in air, allowing the carrier particles to be carried upwards from the bottom of the spraying chamber. The fluidized core particles are then hit by one or more small droplets of fenofibrate dispersion which are ejected from the nozzle. After spraying, the solvent provided on the cores is evaporated to obtain granules of fenofibrate.

The expression “inert carriers” means any excipients, generally water-soluble or water-insoluble, pharmaceutically inert, crystalline or amorphous, in a particulate form, and not leading to a chemical reaction under the operating conditions employed. Combinations of both water-soluble and water-insoluble materials can also be employed.

Examples of water-soluble carrier include, but are not limited to, cellulose derivatives, starch, gums, alginates, polyvinylprrolidone, polyethylene glycol, acrylic acid derivatives, carbohydrate based polymers or any other pharmaceutically acceptable water-soluble materials. Examples of water-insoluble carrier include, but are not limited to, pregelatinized starch, crospovidone, colloidal silicon dioxide, microcrystalline cellulose, cross-linked sodium carboxymethylcellulose, starch, carboxymethylcellulose calcium, calcium carbonate, dibasic calcium phosphate or any other pharmaceutically acceptable water-insoluble materials. Particularly, the inert carrier used in the composition is water-insoluble carrier and more particularly, it is pregelatinised starch.

The composition of the present invention may include one or more other pharmaceutically acceptable excipients that would aid in preparing pharmaceutically acceptable dosage forms like capsule or tablet.

The pharmaceutically acceptable excipients may be one or more of fillers, binders, disintegrants, lubricants, glidants, coloring agents and flavoring agents.

Suitable examples of fillers include, but are not limited to, corn starch, lactose, white sugar, sucrose, sugar compressible, sugar confectioners, glucose, sorbitol, calcium carbonate, calcium phosphate-dibasic, calcium phosphate-tribasic, calcium sulfate, microcrystalline cellulose, silicified microcrystalline cellulose, cellulose powdered, dextrates, dextrins, dextrose, fructose, kaolin, lactitol, mannitol, sorbitol, starch, starch pregelatinized and sucrose.

Examples of binders include, but are not limited to, methyl cellulose, hydroxypropyl cellulose, hydroxypropyl methylcellulose, polyvinylpyrrolidone, gelatin, gum Arabic, ethyl cellulose, polyvinyl alcohol, pullutan, pregelatinized starch, agar, tragacanth, sodium alginate and propylene glycol.

Examples of disintegrants include, but are not limited to, starch, croscarmellose sodium, crospovidone, sodium starch glycolate, or mixtures thereof.

Examples of lubricants and glidants include, but are not limited to, colloidal anhydrous silica, stearic acid, fumaric acid, magnesium stearate, sodium stearyl fumarate, calcium stearate, talc, hydrogenated castor oil, sucrose esters of fatty acids, microcrystalline wax, yellow beeswax, white beeswax, and the like.

The coloring agents of the present invention may be selected from any FDA approved colors for oral use.

The fenofibrate composition may be formulated into various pharmaceutical preparations for oral administration, e.g., in the form of a capsule or a tablet in accordance with any of the conventional procedure known in the field of art, for example, milling, sieving, slugging, kneading, granulating, tabletting, coating, etc. These steps may be carried out in the conventional manner. Most preferably the composition is in capsule form.

The fenofibrate composition prepared by the present invention may be coated with one or more layers comprising film forming agents and/or pharmaceutically acceptable excipients.

The coating layers over the tablet may be applied as solution/dispersion of coating ingredients using any conventional technique known in the prior art such as spray coating in a conventional coating pan or fluidized bed processor or dip coating.

Examples of solvents used for preparing a solution/dispersion of the coating ingredients include, but are not limited to, methylene chloride, isopropyl alcohol, acetone, methanol, ethanol, water, and mixtures thereof.

Examples of film forming agents include, but are not limited to, ethyl cellulose, hydroxypropyl methylcellulose, hydroxypropyl cellulose, methyl cellulose, carboxymethylcellulose, hydroxymethylcellulose, hydroxyethylcellulose, hydroxypropyl methyl phthalate, cellulose acetate, cellulose acetate trimelliatate, cellulose acetate phthalate; waxes such as polyethylene glycol; methacrylic acid polymers such as Eudragit® RL and RS; or mixture thereof. Alternatively, commercially available coating compositions comprising film-forming polymers marketed under various trade names, such as Opadry® may also be used for coating.

The fenofibrate composition according to the present invention may be used to treat hyperlipidaemic or dyslipidimic conditions. The composition according to the present invention comprising fenofibrate may be administered in combination with other medicines, for example, other antilipidaemic agents.

The present invention is illustrated below by reference to the following example. However, one skilled in the art will appreciate that the specific methods and results discussed are merely illustrative of the invention, and not to be construed as limiting the invention.

### Example

### Preparation of Fenofibrate Capsules

### Composition:

| Ingredients | Percent w/w |
|---|---|
| Fenofibrate | 42.48 |
| Sodium Lauryl Sulphate | 0.85 |
| Hydroxypropyl Methylcellulose | 17.16 |
| Simethicone Emulsion | 0.27 |
| Purified Water | q.s. |
| Pregelatinized Starch | 37.28 |
| Talc | 1.31 |
| Sodium stearyl Fumarate | 0.65 |

### Procedure:

1. Fenofibrate, sodium lauryl sulphate, hydroxypropyl methylcellulose and simethicone were added to water and stirred to get a homogenized dispersion.
2. The dispersion of step 1 was sprayed over the pregelatinized starch and dried, to form fenofibrate granules.
3. The granules of step 2 were sifted and blended with talc and sodium stearyl fumarate.
4. The mixture obtained in step 3 was filled into capsules.

While there has been shown and described what are the preferred embodiments of the invention, one skilled in the pharmaceutical formulation art will appreciate that various modifications in the formulations and process can be made without departing from the scope of the invention as it is defined by the appended claims.

We claim:

1. A fenofibrate composition in the form of capsules or tablets, comprising:
(i) fenofibrate, in a concentration from 25 to 50% by weight of the composition,
(ii) a surfactant, in a concentration from 0.05 to 5% by weight of the composition,
(iii) a hydrophilic polymer, in a concentration from 15 to 20% by weight of the composition, and
(iv) one or more anti-foaming agents, in a concentration from 0.05 to 5% by weight of the composition.

2. The composition according to claim 1, wherein the fenofibrate is in micronized form having d_{0.5} between 1 μm to 20 μm and d_{0.9} between 2 μm to 40 μm.

3. The composition according to claim 1, wherein the surfactant comprises one or more of anionic, cationic, zwitterionic and nonionic surfactants.

4. The composition according to claim 1, wherein the hydrophilic polymer comprises one or more of starch, gums, alginates, polysaccharides, polyvinylprrolidone, polyethylene glycol, acrylic acid derivatives, cellulose derivatives like hydroxypropyl cellulose, hydroxypropyl methylcellulose, hydroxyethylcellulose, hydroxymethylcellulose, carboxymethylcellulose, methylcellulose, sodium carboxy methylcellulose, and mixtures thereof.

5. The composition according to claim 1, wherein the anti-foaming agents are selected from one or more of simethicone, polyglycol, silicon oil, and mixtures thereof.

6. A process for preparing a fenofibrate composition comprising the steps of:
(i) preparing a homogenized dispersion comprising fenofibrate, a surfactant, a hydrophilic polymer and one or more anti-foaming agent,
(ii) spraying the dispersion of step (i) over one or more inert carriers to form granules,
(iii) drying the granules of step (ii) and optionally, blending with one or more pharmaceutically acceptable excipients,
(iv) filling the mixture of step (iii) into capsules or compressing into tablets.

7. The process according to claim 6, wherein the inert carriers comprise one or more pharmaceutically acceptable materials of water-soluble, water-insoluble and combinations thereof.

8. The process according to claim 6, wherein the pharmaceutically acceptable excipients comprise one or more of fillers, binders, disintegrants, lubricants, glidants, coloring agents and flavoring agents.

9. The process according to claim 6, wherein the composition is optionally further coated with one or more film forming agents.

10. The method of treating hyperlipidaemic or dyslipidimic conditions in a patient by administering the fenofibrate composition according to claim 1.

11. The method according to claim 10, wherein the fenofibrate composition further comprises one or more other antilipidaemic agents.
